(19) United States
(12) Patent Application Publication
(10) Pub. No.: US 2024/0173484 A1
(43) Pub. Date: May 30, 2024

(54) INJECTION DEVICE WITH INJECTION COMPLETION INDICATOR

(71) Applicants: PULSE NEEDLEFREE SYSTEMS, INC., LENEXA, KS (US); OTSUKA PHARMACEUTICAL CO., LTD., TOKYO (JP)

(72) Inventor: MIKHAIL ZOLOTUKHIN, SHAWNEE, KS (US)

(21) Appl. No.: 18/284,555

(22) PCT Filed: Mar. 31, 2022
(86) PCT No.: PCT/US2022/022700

§ 371 Date: Sep. 28, 2023

Related U.S. Application Data
(60) Provisional application No. 63/169,277, filed on Apr. 1, 2021.

(51) Int. Cl. A61M 5/31; A61M 5/32

(52) U.S. Cl. CPC A61M 5/3137; A61M 5/32; A61M 2205/583

(57) ABSTRACT

In an aspect, the present invention is directed to an injection device. The injection device includes a pneumatic piston chamber and a pneumatic piston slidably positioned within the pneumatic piston chamber; a dose chamber and an injectate piston slidably positioned within the dose chamber; and an injection completion indicator comprising a window into the piston chamber or dose chamber, wherein the injection completion indicator is aligned with a portion of the pneumatic piston chamber or the dose chamber. In another aspect, the present invention is directed to a method of administering an injection to a subject using the injection device of the invention.

### CROSS-REFERENCE TO RELATED APPLICATIONS

This application is based on and claims priority to U.S. Provisional Application Ser. No. 63/169,277 filed on Apr. 1, 2021, which is hereby incorporated herein by reference.

### BACKGROUND OF THE INVENTION

### 1. Field of the Invention

The present invention relates to the field of injection devices.

### 2. Description of Related Art

Historically, plant pests and diseases have been controlled by mass application of pesticides. However, many pesticides contain nicotine. Nicotine-based pesticides have been shown to be a cause in the decline of bee populations and are being restricted in certain jurisdictions. Concerns over the environmental and health impacts of conventional pesticides have led to the research and development of more natural pesticides or biocontrol measures that are delivered to the plant in small doses via injection.

In developing alternatives to mass application of conventional pesticides for the control of plant bacteria, researchers have developed virulent bacteriophages that serve as biocontrol agents for diseases. One such disease is Pierce's Disease, which impacts grapevines and other plants, and is spread plant to plant via insects. While conventional pesticides were used to control the insects that transmit plant disease between plants, bacteriophages seek to limit the impact of the bacteria once the bacteria is present within the plant. Unlike conventional pesticides, however, bacteriophages cannot effectively be applied to the surface of the plant and must instead be injected into the plant to be effective.

When injecting vascular plants for certain applications, such as bacteriophages, it is desirable to deliver the injectate into the xylem of the plant. Xylem is plant vascular tissue that transports water and nutrients from the plant's roots to its stems and leaves. As such, injections into the xylem can allow the injectate to be transported and exposed, via the xylem's natural pathways, to a large portion of the plant's tissues. Injections into the xylem may also be desirable when the injectate is intended to affect bacteria that are present in the plant's xylem tissues. Known applications for injections into the xylem tissue are prophylactics and treatments related to the plant pathogen bacteria Xylella fastidiosa and Xanthomonas, commonly known as Pierce's Disease in viticulture.

When injecting liquid into the xylem of grapevines, it is important that the entire set dose is administered such that the injection is complete. However, this can be challenging if not impossible.

### BRIEF SUMMARY OF THE INVENTION

In an aspect, the present invention is directed to an injection device. The injection device includes a pneumatic piston chamber and a pneumatic piston slidably positioned within the pneumatic piston chamber: a dose chamber and an injectate piston slidably positioned within the dose chamber: and an injection completion indicator comprising a window into the piston chamber or dose chamber, wherein the injection completion indicator is aligned with a portion of the pneumatic piston chamber or the dose chamber. In certain embodiments, the injection device includes an indicator portion of the pneumatic piston or injectate piston. The indicator portion is an outer surface of the pneumatic piston or injectate piston that is configured to align with and thus be visible through the injection completion indicator only when the pneumatic piston or injectate piston is in a post-injection position. In certain embodiments, the indicator portion is a colored indicator ring positioned, formed, and/or painted on an outer surface of the pneumatic piston or injectate piston.

In another aspect, the present invention is directed to a method of administering an injection to a subject using the injection device of the invention. The method includes the following steps: pressing a tip of a needle into a subject to be injected: injecting an injectate into the subject to be injected: and observing the dose completion indicator to ascertain whether the pneumatic piston or injectate piston has reached its most distal position within the pneumatic piston chamber or the dose chamber. In embodiments which include an indicator portion of the pneumatic piston or injectate piston, the observing step requires observing the dose completion indicator to ascertain whether the indicator portion of the pneumatic piston or injectate piston is visible through the injection completion indicator.

Additional aspects of the invention, together with the advantages and novel features appurtenant thereto, will be set forth in part in the description which follows, and in part will become apparent to those skilled in the art upon examination of the following, or may be learned from the practice of the invention. The objects and advantages of the invention may be realized and attained by means of the instrumentalities and combinations particularly pointed out in the appended claims.

### BRIEF DESCRIPTION OF THE DRAWINGS

FIG. 1 is a perspective-side view of an exemplary injection device in the pre-injection position.

FIG. 2 is a cross-sectional view of the injection device of FIG. 1 in the pre-injection position.

FIG. 3 is a cross-sectional view of the injection device of FIG. 1 in the post-injection position.

FIG. 4 is a perspective-side view of the injection device in the post-injection position.

### DETAILED DESCRIPTION OF PREFERRED EMBODIMENT

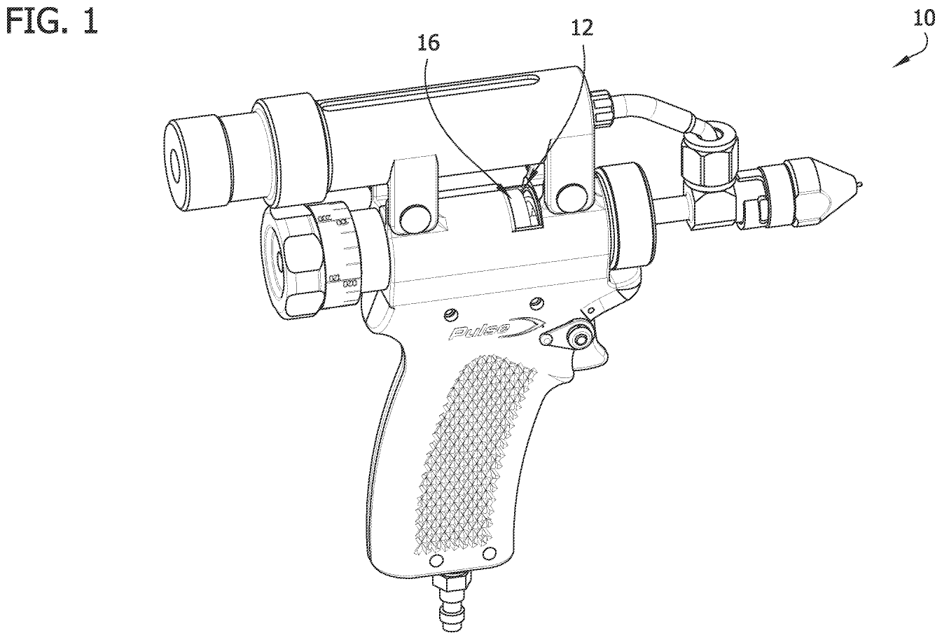

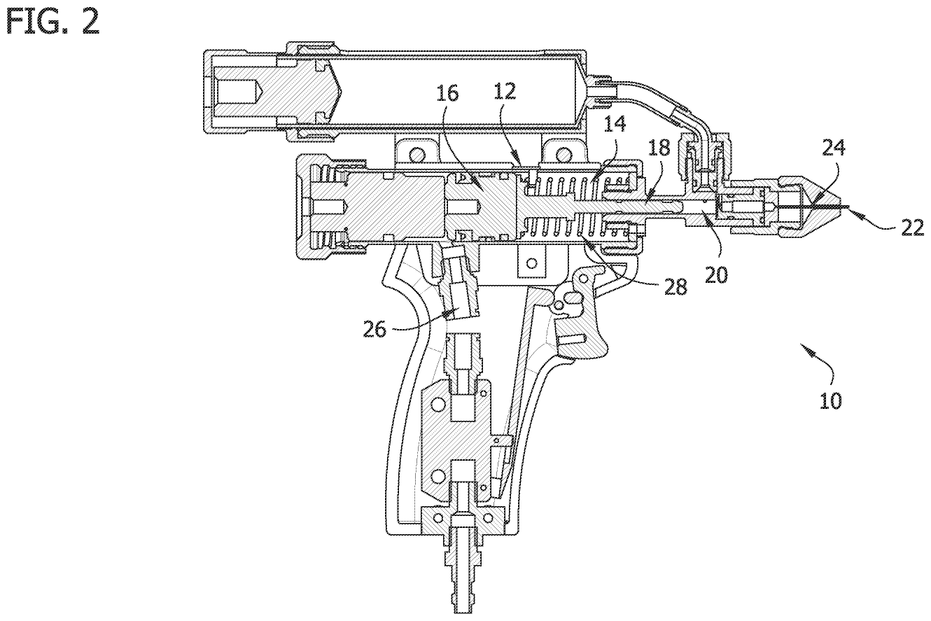

Certain aspects of the invention will now be described with respect to the exemplary embodiments depicted in the figures, but are not limited to such embodiments. In an aspect, the present invention is directed to an injection device with an injection completion indicator, which is an observation window. The injection completion indicator allows a user to ascertain whether an injection is complete. In other words, the injection completion indicator allows a user to ascertain whether a set dose has been administered in its entirety. In certain embodiments, the injection completion indicator is aligned with a portion of a piston chamber, such that the injection completion indicator allows a user to observe whether the injection has been completed. In certain embodiments, the injection completion indicator is aligned with a distal portion of the piston chamber to allow a user to observe whether the piston has reached its most distal position within the piston chamber after completion of the injection. In certain embodiments, the outer surface of the pneumatic piston of the injection device may further comprise an indicator portion that is configured to align with and thus be visible through the injection completion indicator 12 only when an injection is complete. An exemplary embodiment of a grapevine injection device comprising the injection completion indicator of the present invention is depicted in FIGS. 1 and 2.

Referring to FIG. 2, injection device 10 includes injection completion indicator 12, pneumatic piston chamber 14, pneumatic piston 16, injectate piston 18, dose chamber 20, and needle 22. Pneumatic piston 16 is slidably positioned within pneumatic piston chamber 14. Injectate piston 18 is slidably positioned within dose chamber 20. A proximal portion of injectate piston 18 is attached to a distal portion of pneumatic piston 16. Accordingly, movement of pneumatic piston 16 in the distal direction in turn causes injectate piston 18 to move in the distal direction. Similarly, movement of injectate piston 18 in the proximal direction in turn causes pneumatic piston 16 to move in the proximal direction. Injection completion indicator 12 is aligned with a distal portion of pneumatic piston chamber 14 so that the user can ascertain whether pneumatic piston 16 is in its most distal position.

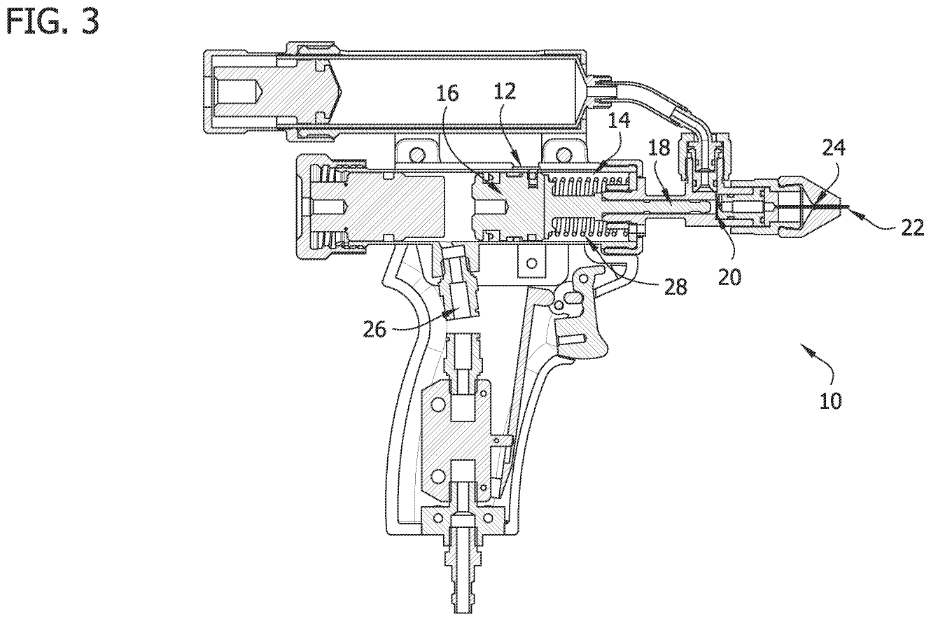

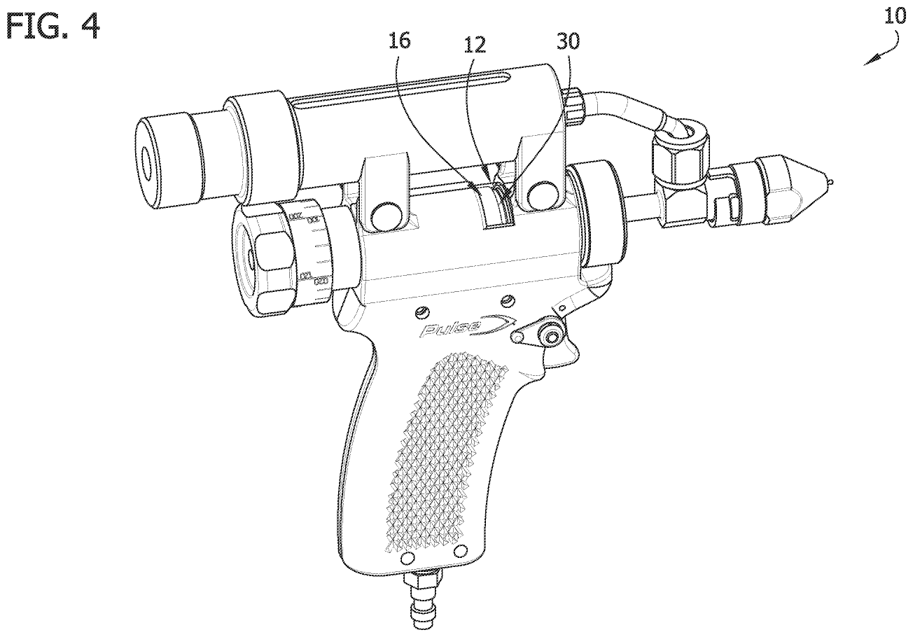

Pneumatic piston 16 and injectate piston 18 are in their most proximal positions in the pre-injection positions shown in FIGS. 1 and 2. Pneumatic piston 16 and injectate piston 18 are in their most distal positions in the post-injection positions shown in FIGS. 3 and 4. The movement of the pneumatic piston 16 and injectate piston 18 from the pre-injection positions to the post-injection positions causes any injectate (not shown) present in dose chamber 20 to be forced into a longitudinal channel 24 of needle 22 where the injectate may then flow into a subject to be injected from one or more openings of needle 22. Accordingly, pneumatic piston 16 and injectate piston 18 are in the most distal post-injection positions when an injection has been completed (i.e., when a set dose has been administered in its entirety). Injection completion indicator 12, aligned with the distal end of pneumatic piston chamber 14, thus allows the user to observe whether pneumatic piston 16 is in its post-injections position after an injection has been completed.

After the injection has been completed, pneumatic piston 16 and injectate piston 18 are returned to their pre-injection positions. In certain embodiments, this is achieved by terminating pressure supplied to pneumatic piston and/or injectate piston during injection, allowing return spring 28 to decompress and to force pneumatic piston 16 and injectate piston 18 to move to the pre-injection positions. More specifically, in order to move pneumatic piston 16 and injectate piston 18 from the pre-injection positions to the post-injection positions, pressurized fluid (not shown) (e.g., air) can be supplied to pneumatic piston chamber 14 via gas supply line 26 where it is then directed to proximal portions of pneumatic piston 16. This results in the application of force to a proximal portion of pneumatic piston 16 sufficient to cause the compression of return spring 28 and to force pneumatic piston 16 and injectate piston 18 to move to the post-injection positions. In order to move pneumatic piston 16 and injectate piston 18 from the post-injection positions to the pre-injection positions, the supply of pressurized fluid to pneumatic piston chamber via gas supply line 26 is terminated. This allows return spring 28 to decompress and to force pneumatic piston 16 and injectate piston 18 to move to the pre-injection positions.

Referring to FIGS. 1 and 2, pneumatic piston chamber 14 includes an injection completion indicator 12, which is an indication or observation window. Injection completion indicator 12 is at least partially transparent such that portions of pneumatic piston chamber 14 and the components therein may be visible to an operator. Injection completion indicator 12 may be formed integrally with pneumatic piston chamber 14. Alternatively, injection completion indicator 12 may be positioned within or overlap with an opening in pneumatic piston chamber 14. Injection completion indicator 12 may be formed from any transparent or partially transparent material known to those of ordinary skill in the art which is suitable for use in constructing a portion of a pneumatic piston chamber of an injection device.

Injection completion indicator 12 allows a user to ascertain whether injection device 10 has completed an injection such that pneumatic piston 16 and injectate piston 18 are in the post-injection positions. Referring to FIG. 4, in certain embodiments, when pneumatic piston 16 and injectate piston 18 are in the post-injection positions, an indicator portion 30 of the outer surface of pneumatic piston 16 is aligned with and thus visible through injection completion indicator 12. When pneumatic piston 16 and injectate piston 18 are in any position other than the post-injection positions, including but not limited to the pre-injection positions (shown in FIGS. 1 and 2), the indicator portion 30 of the outer surface of pneumatic piston 16 is not aligned with and thus is not visible through injection completion indicator 12. Indicator portion 30 may be any readily identifiable portion of pneumatic piston 16. For example, indicator portion may be a colored (e.g., red) indicator ring positioned, formed, and/or painted on the outer cylindrical surface of pneumatic piston 16.

In certain embodiments, the injection completion indicator of the present invention may be included on dose chamber 20 instead of pneumatic piston chamber. In such embodiments, when pneumatic piston 16 and injectate piston 18 are in the post-injection positions, an indicator portion of the outer surface of injectate piston 18 is aligned with and thus visible through injection completion indicator 12. When pneumatic piston 16 and injectate piston 18 are in the pre-injection positions, the indicator portion of the outer surface of injectate piston 18 is not aligned with and thus is not visible through injection completion indicator 12.

As will be readily understood to those of ordinary skill in the art, a variety of injection devices may be configured or modified to be injection devices of the present invention. For example, the grape vine injection devices shown and described in PCT/US2020/028172 (filed on Apr. 15, 2020) and U.S. Provisional Application No. 62/835,700 (filed on Apr. 18, 2019) may be configured or modified to include an injection completion indicator of the present invention. The disclosures of PCT/US2020/028172 (filed on Apr. 15, 2020) and U.S. Provisional Application No. 62/835,700 (filed on Apr. 18, 2019) are incorporated by reference in their entireties herein.

In another aspect, the present invention is directed to a method of administering an injection to a subject with the injection device of the present invention. The method of administering an injection to a subject includes the following steps: pressing a tip of a needle into a subject to be injected: injecting an injectate into the subject to be injected: and observing the dose completion indicator to ascertain whether the pneumatic piston or injectate piston has reached its most distal position within the pneumatic piston chamber or the dose chamber. In embodiments which include an indicator portion of the pneumatic piston or injectate piston, the observing step requires observing the dose completion indicator to ascertain whether the indicator portion of the pneumatic piston or injectate piston is visible through the injection completion indicator.

From the foregoing it will be seen that this invention is one well adapted to attain all ends and objectives herein-above set forth, together with the other advantages which are obvious and which are inherent to the invention.

Since many possible embodiments may be made of the invention without departing from the scope thereof, it is to be understood that all matters herein set forth or shown in the accompanying drawings are to be interpreted as illustrative, and not in a limiting sense.

While specific embodiments have been shown and discussed, various modifications may of course be made, and the invention is not limited to the specific forms or arrangement of parts and steps described herein, except insofar as such limitations are included in the following claims. Further, it will be understood that certain features and subcombinations are of utility and may be employed without reference to other features and subcombinations. This is contemplated by and is within the scope of the claims.

1. An injection device, comprising:
a pneumatic piston chamber and a pneumatic piston slidably positioned within the pneumatic piston chamber;
a dose chamber and an injectate piston slidably positioned within the dose chamber; and
an injection completion indicator comprising a window into the piston chamber or dose chamber, wherein the injection completion indicator is aligned with a portion of the pneumatic piston chamber or the dose chamber.

2. The injection device of claim 1, further comprising: an indicator portion of the pneumatic piston or injectate piston, wherein the indicator portion is an outer surface of the pneumatic piston or injectate piston, and wherein the indicator portion is configured to align with and thus be visible through the injection completion indicator only when the pneumatic piston or injectate piston is in a post-injection position.

3. The injection device of claim 2, wherein the indicator portion is a colored indicator ring positioned, formed, and/or painted on an outer surface of the pneumatic piston or injectate piston.

4. A method of administering an injection to a subject using the injection device of claim 1, comprising:
pressing a tip of a needle into a subject to be injected;
injecting an injectate into the subject to be injected;
and observing the injection completion indicator to ascertain whether the pneumatic piston or injectate piston has reached its most distal position within the pneumatic piston chamber or the dose chamber.

5. A method of administering an injection to a subject using the injection device of claim 2, comprising:
pressing a tip of a needle into a subject to be injected;
injecting an injectate into the subject to be injected; and
observing the injection completion indicator to ascertain whether the indicator portion of the pneumatic piston or injectate piston is visible through the injection completion indicator.
